(19) United States
(12) Patent Application Publication
(10) Pub. No.: US 2024/0240147 A1
(43) Pub. Date: Jul. 18, 2024

(54) CHIMERIC ANTIGEN RECEPTOR-MODIFIED GRANULOCYTE-MACROPHAGE PROGENITORS FOR CANCER IMMUNOTHERAPY

(71) Applicant: University of Southern California, Los Angeles, CA (US)

(72) Inventors: Qi-Long Ying, Los Angeles, CA (US); Shi Yue, Los Angeles, CA (US); Xueyuan Jing, Los Angeles, CA (US)

(21) Appl. No.: 18/561,889

(22) PCT Filed: May 19, 2022
(86) PCT No.: PCT/US2022/030109

§ 371 Date: Nov. 17, 2023

Related U.S. Application Data
(60) Provisional application No. 63/190,387, filed on May 19, 2021.

(51) Int. Cl. C12N 5/0787; A61K 39/00; A61P 35/02; C07K 14/705; C12N 5/0786; C12N 5/0789; C12N 15/86

(52) U.S. Cl. CPC C12N 5/0642; A61K 39/4614; A61K 39/4631; A61P 35/02; C07K 14/70503; C12N 5/0645; C12N 5/0647; C12N 15/86; C07K 2319/03; C12N 2500/25; C12N 2500/36; C12N 2500/46; C12N 2501/125; C12N 2501/22; C12N 2501/415; C12N 2501/727; C12N 2506/11; C12N 2510/00; C12N 2740/15041

(57) ABSTRACT

The disclosure provides methods to genetically engineer granulocyte-macrophage progenitors (GMPs) to express a chimeric antigen receptor (CAR), and uses thereof, including for cancer immunotherapy.

### CROSS REFERENCE TO RELATED APPLICATIONS

This application claims priority under 35 U.S.C. § 119 from Provisional Application Ser. No. 63/190,387, filed May 19, 2021, the disclosure of which is incorporated herein by reference.

### TECHNICAL FIELD

The disclosure provides methods to genetically engineer granulocyte-macrophage progenitors (GMPs) to express a chimeric antigen receptor (CAR), and uses thereof, including for cancer immunotherapy.

### BACKGROUND

Granulocytes, macrophages, and dendritic cells are the essential components of the innate immune system in humans. They are the first line of defense against pathogens and also play a central role in maintaining the homeostasis of our body and preventing various diseases including infection, metabolic diseases and cancer. These cells originate from a common progenitor in the bone marrow, the granulocyte-macrophage progenitor (GMP).

### SUMMARY

The granulocyte-monocyte progenitor (GMP) is the common progenitor for granulocytes and macrophages, the two major components of the innate immune system. The inability to perform the long-term expansion of GMPs and their derivatives has greatly limited the therapeutic applications of these immune cells. In the studies presented herein, it was shown that homogeneous GMPs can be exponentially expanded long-term in fully defined conditions. The expanded GMPs retained key features of GMPs, including the ability to differentiate into functional granulocytes and macrophages. Transplantation of expanded GMPs effectively prevented bacterial infection in immunodeficient mice. Furthermore, the expanded GMPs can be genetically engineered to produce macrophages that specifically phagocytize cancer cells. The methods and compositions described herein allowed for exponential expansion and genetically engineered GMPs. The GMPs made by the methods and compositions of the disclosure are useful for the development of immunotherapies to treat a wide range of diseases, especially infectious diseases and cancers.

In a particular embodiment, the disclosure provides a method to genetically engineer granulocyte-macrophage progenitors (GMPs) to express a chimeric antigen receptor (CAR) comprising: introducing a vector comprising a CAR into GMPs to form GMPs that express CAR (CAR-GMPs); expanding and culturing the CAR-GMPs for multiple passages in defined culture conditions to generate a population of CAR-GMPs; and inducing the population of CAR-GMPs to differentiate into granulocytes, macrophages or dendritic cells in vitro, wherein the granulocytes, macrophages or dendritic cells express CAR. In a further embodiment, the GMPs are obtained from stem cells. In yet a further embodiment, the stem cells are hematopoietic stem cells. In another embodiment, the hematopoietic stem cells are isolated from the bone marrow of a subject. In yet another the subject is a mammalian subject. In a further embodiment, the subject is a human patient. In yet a further embodiment, the CAR comprises an extracellular domain capable of binding to an antigen, a transmembrane domain and at least one intracellular domain that is designed to increase the anti-tumor activities of granulocytes, macrophages and dendritic cells by increasing their phagocytosis and/or proinflammatory cytokines secretion. In another embodiment, the vector is a viral vector. In yet another embodiment, the viral vector can be replicating or non-replicating, and can be an adenoviral vector, an adeno-associated virus (AAV) vector, a measles vector, a herpes vector, a retroviral vector, a lentiviral vector, a rhabdoviral vector, a reovirus vector, a Seneca Valley Virus vector, a poxvirus vector, a parvovirus vector, or an alphavirus vector. In a certain embodiment, the viral vector is a lentiviral vector. In another embodiment, the defined culture conditions include culturing the CAR-GMPs in a culture medium comprising: (i) a growth factor, (ii) a B-Raf kinase inhibitor, and (iii) a Wnt activator and/or a GSK-3 inhibitor, wherein the CAR-GMPs remain substantially morphologically unchanged after undergoing multiple cell passages and/or clonal expansion. In a further embodiment, the culture medium comprises DMEM/F12 and Neural Basal Medium. In yet a further embodiment, the culture medium comprises DMEM/F12 and Neural Basal Medium in a ratio of about 5:1 to about 1:5. In another embodiment, the culture medium comprises DMEM/F12 and Neural Basal Medium in a ratio of about 1:1. In yet another embodiment, the culture medium comprises one or more supplements selected from insulin, transferrin, bovine serum albumin (BSA) fraction V, putrescine, sodium selenite, DL-α tocopherol, and/or linolenic acid. In a further embodiment, the culture medium is supplemented with insulin, transferrin, BSA fraction V, putrescine, sodium selenite, DL-α tocopherol, and linolenic acid. In a certain embodiment, the growth factor is stem cell factor (SCF). In another embodiment, the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof. In yet another embodiment, the Wnt activator is selected from the group consisting of SKL 2001, BML-284, WAY 262611, CAS 853220-52-7, QS11 and any combination thereof. In a further embodiment, the GSK-3 inhibitor is selected from the group consisting of CHIR99021, CHIR98014, SB216763, BIO, A1070722, AR-A014418 and any combination thereof. In another embodiment, the defined culture conditions include culturing the CAR-GMPs in a culture medium comprising: (i) a growth factor; (ii) a B-Raf kinase inhibitor; (iii) an agent that inhibits the mitogen-activated kinase interacting protein kinases 1 and 2 (Mnk1/2); (iv) an agent that inhibits the PI3K pathway; (v) optionally, one or more serum components; wherein the CAR-GMPs remain substantially morphologically unchanged after undergoing multiple cell passages and/or clonal expansion. In a further embodiment, the culture medium comprises DMEM/F12 and Neural Basal Medium. In yet a further embodiment, the culture medium comprises DMEM/F12 and Neural Basal Medium in a ratio of about 5:1 to about 1:5. In another embodiment, the culture medium comprises DMEM/F12 and Neural Basal Medium in a ratio of about 1:1. In yet another embodiment, the culture medium comprises one or more supplements selected from insulin, transferrin, bovine serum albumin (BSA) fraction V, putrescine, sodium selenite, DL-α tocopherol, and/or linolenic acid. In a further embodiment, the culture medium is supplemented with insulin, transferrin, BSA fraction V, putrescine, sodium selenite, DL-α tocopherol, and linolenic acid. In yet a further embodiment, the growth factor is stem cell factor (SCF). In another embodiment, the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof. In a further embodiment, the agent that inhibits Mnk1/2 is selected from the group consisting of CGP-57380, cercosporamide, BAY 1143269, tomivosertib, ETC-206, SLV-2436 and any combination thereof. In yet a further embodiment, the agent that inhibits PI3K pathway is selected from the group consisting of 3-methyladenine, LY294002, alpelisib, wortmannin, quercetin, hSMG-1 inhibitor 11j, zandelisib, alpelisib hydrochloride, idelalisib, buparlisib, copanlisib, IPI549, dactolisib, pictilisib, SAR405, duvelisib, fimepinostat, GDC-0077, PI-103, YM-20163, PF-04691502, Taselisib, omipalisib, samotolisib, isorhamnetin, ZATK474, parsaclisib, rigosertib, AZD8186, GSK2636771, disitertide, TG100-115, AS-605240, PI3K-IN-1, dactolisib tosylate, gedatolisib, TGX-221, umbralisib, AZD 6482, serabelisib, bimiralisib, apitolisib, alpha-linolenic acid, Vps34-PIK-III, PIK-93, Vps34-IN-1, CH5132799, leniolisib, voxtalisib, GSK1059615, sonolisib, PKI-402, PI4KIIIbeta-IN-9, HS-173, BGT226 maleate, pictilisib dimethane sulfonate, VS-5584, IC-87114, quercetin dihydrate, CNX-1351, SF2523, GDC-0326, seletalisib, acalisib, SAR-260301, ZAD-8835, GNE-317, AMG319, nemiralisib, IITZ-01, PI-103 hydrochloride, oroxin B, pilaralisib, AS-252424, cpanlisib dihydrochloride, AMG 511, disitertide TFA, PIK-90, tenalisib, esculetin, CGS 15943, GNE-477, PI-3065, A66, AZD3458, ginsenoside Rk1, sophocarpine, buparlisib hydrochloride, Vps34-IN-2, linperlisib, arnicolide D, KP372-1, CZC24832, PF-4989216, (R)-Duvelisib, PQR530, P11δ-IN-1, umbralisib hydrochloride, MTX-211, PI3K/mTOR Inhibitor-2, LX2343, PF-04979064, polygalasaponin F, glaucocalyxin A, NSC781406, MSC2360844, CAY10505, IPI-3063, TG 100713, BEBT-908, PI-828, brevianamide F, ETP-46321, PIK-294, SRX3207, sophocarpine monohydrate, AS-604850, desmethylglycitein, SKI V, WYE-687, NVP-QAV-572, GNE-493, CAL-130 hydrochloride, GS-9901, BGT226, IHMT-PI3Kδ-372, PI3Kα-IN-4, parsaclisib hydrochloride, PF-06843195, PI3K-IN-6, (S)-PI3Kα-IN-4, PI3K(gama)-IN-8, BAY1082439, CYH33, PI3Kγ inhibitor 2, PI3Kδ inhibitor 1, PARP/PI3K-IN-1, LAS191954, PI3K-IN-9, CHMFL-PI3KD-317, PI3K/HDAC-IN-1, MSC2360844 hemifumarate, PI3K-IN-2, PI3K/mTOR Inhibitor-1, PI3Kδ-IN-1, euscaphic acid, KU-0060648, AZD 6482, WYE-687 dihydrochloride, GSK2292767, (R)-Umbralisib, PIK-293, idelalisib D5, PIK-75, hirsutenone, quercetin D5, PIK-108, hSMG-1 inhibitor 11e, PI3K-IN-10, NVP-BAG956, PI3Kγ inhibitor 1, CAL-130, ON 146040, PI3kδ inhibitor 1, PI3Kα/mTOR-IN-1, and any combination thereof. In another embodiment, the CAR-GMPs are induced to differentiate into macrophages comprising: culturing the CAR-GMPs with a macrophage differentiation medium comprising macrophage colony-stimulating factor (MCSF), wherein the macrophages express CAR. In yet another embodiment, the macrophage differentiation medium comprises RPMI 1640, fetal bovine serum (FBS) and MCSF. In an alternate embodiment, the method further comprises differentiating the CAR-GMPs into granulocytes comprising: culturing the GMPs with a granulocyte differentiation medium comprising granulocyte colony-stimulating factor (GCSF), wherein the granulocytes express CAR. In a further embodiment, the granulocyte differentiation medium comprises RPMI 1640, FBS and GCSF.

In a certain embodiment, disclosure also provides for macrophages that express CAR made by a method of the disclosure.

In a particular embodiment, disclosure further provides for granulocytes that express CAR made by a method of the disclosure.

In another embodiment, the disclosure provides an immunotherapy method for treating a subject having cancer with macrophages or granulocytes that express CAR: administering a composition comprising macrophages that express CAR made by a method of the disclosure or granulocytes that express CAR made by a method of the disclosure to the subject having cancer. In a further embodiment, the composition is administered intravenously or inter-tumoral. In yet a further embodiment, macrophages or granulocytes are obtained from GMPs from stem cells of the subject to be treated with the immunotherapy method. In another embodiment, the subject has a cancer selected from adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In yet another embodiment, the immunotherapy method further comprises administering one or more anticancer agents to the subject having cancer.

### DESCRIPTION OF DRAWINGS

FIG. 1A-E demonstrates that αCD19 CAR-macrophages generated from engineered SCF/2i GMPs effectively phagocytize human B-ALL cells. (A) SCF/2i GMPs were electroporated with GFP mRNA or transduced with GFP lentivirus (pSin-GFP). GFP expression was analyzed by fluorescent microscope (upper panel) and flow cytometry (lower panel) 48 hours after transfection. Flow cytometry data are represented as mean±SD from five independent experiments. (B) SCF/2i GMPs derived from CAG-Cas9-GFP mice were electroporated with control or GFP sgRNA, GFP expression was analyzed by flow cytometry 48 hours after electroporation. Data are represented as mean±SD from three independent experiments. (C) Schematic diagrams showing the structures of CarP-RFP, CarPFc-19-RFP, and CarPzFc-19-RFP. (D) SCF/2i GMPs were transduced with CarP-RFP or CarPzFc19-RFP lentivirus and RFP-positive GMPs were sorted and further expanded in SCF/2i. 1×10⁵ macrophages derived from RFP-positive GMPs were co-cultured with 1×10⁶ GFP-positive human B-ALL cells pretreated with or without anti-CD47 antibody. Cells were washed with PBS one hour after co-culture and phase-contrast and fluorescent images were taken. (E) Cells in (D) were trypsinized and GFP and RFP expression was analyzed by flow cytometry. The percentages of phagocytotic macrophages were quantified. Data are represented as mean±SD from three independent experiments.

FIG. 2A-C shows the expansion, differentiation, and genetic engineering of GMPs. (A) Phagocytosis analysis of GMP-derived macrophages by incubating with GFP-labeled E. coli for one hour. Representative phase-contrast and fluorescent images showing the GFP-labeled bacteria engulfed by macrophages and a representative plot of flow cytometry analysis of GMP-derived macrophages incubated with (red) or without (blue) GFP-labeled bacteria. Flow cytometry data are represented as mean±SD from three independent experiments. (B) Differentiated cells were plated into 96-well plates at a density of 2×10⁴ cells/well and stimulated with or without 500 ng/ml LPS for 6 hours, after which cytokine secretion in the supernatants was measured by ELISA. Data are represented as mean±SD from three independent experiments. (C) GMPs expanded in modified SCF/2i were transduced with CarP-RFP or human CarPzFc19-RFP lentivirus. RFP-positive GMPs were sorted and further expanded in modified SCF/2i. Macrophages derived from RFP-positive GMPs were plated into 24-well plates at a density of 1×10⁵ cells/well and cultured in DMEM/10% FBS overnight, after which 1×10⁶ GFP-positive human B-ALL cells pretreated with or without anti-CD47 antibody were added to each well. One hour after co-culture, cells were washed with PBS and trypsinized and GFP and RFP expression was analyzed by flow cytometry. The percentages of phagocytotic macrophages were quantified. Data are represented as mean±SD from three independent experiments.

FIG. 3A-D demonstrates phagocytosis of human B-ALL cells by genetically engineered SCF/2i GMP-derived macrophages, related to FIG. 1. (A) SCF/2i mouse GMPs were transduced with CarP-RFP or CarPFc19-RFP lentivirus and RFP-positive cells were sorted and expanded in SCF/2i. Macrophages derived from RFP-positive mouse GMPs were plated into 24-well plates at a density of 1×10⁵ cells/well and cultured in DMEM/10% FBS overnight, after which 1×10⁶ GFP-positive human B-ALL cells were added to each well. One hour after coculture, cells were washed with PBS and trypsinized and GFP and RFP expression was analyzed by flow cytometry. The percentages of phagocytotic macrophages were quantified. Data are represented as mean±SD from three independent experiments. (B) Time-lapse images showing the process of phagocytosis of CarPzFc19-RFP-expressing macrophages at different time points. Time is shown in minutes. Arrows point to the GFP positive B-ALL cells before and after being phagocytized. Images were extracted from Video. (C) SCF/2i mouse GMPs were transduced with αHER2 CarPzFc19-RFP lentivirus. RFP-positive GMPs were sorted and expanded in SCF/2i. Macrophages derived from RFP-positive GMPs were plated into 24-well plates at a density of 1×10⁵ cells/well and cultured in DMEM/10% FBS overnight, after which 1×10⁶ GFP-positive SK-BR-3 cells were added to each well. One hour after co-culture, phase-contrast and fluorescent images were taken. (D) SCF/2i GMPs were transduced with αCD19 CarPzFc19-RFP or αHER2 CarPzFc19-RFP lentivirus and RFP-positive GMPs were sorted and further expanded in SCF/2i. 1×10⁵ macrophages expressing αCD19 CarPzFc19-RFP or αHER2 CarPzFc19-RFP were co-cultured with GFP-positive human B-ALL cells or SKBR-3 cells. One hour after co-culture, cells were washed with PBS and trypsinized and GFP and RFP expression was analyzed by flow cytometry. The percentages of phagocytotic macrophages were quantified. Data are represented as mean±SD from three independent experiments.

FIG. 4A-B demonstrates that αCD19 CAR-macrophages derived from engineered GMPs effectively phagocytize human B-ALL cells, related to FIG. 2C. (A) GMPs were expanded in the modified SCF/2i and transduced with human CarPzFc19-RFP (h CarPzFc19-RFP) lentivirus. Macrophages derived from hCarPzFc19-RFP-expressing GMPs were co-cultured with GFP-labeled human B-ALL cells. One hour after co-culture, Phase-contrast and fluorescent images were taken. (B) Sequential fluorescent images of hCarPzFc19-RFP-expressing macrophages co-cultured with GFP labeled human B-ALL cells pre-incubated with anti-CD47 antibody.

FIG. 5A-D shows transplantation of αCD19 CAR-GMPs attenuates leukemia cells in mice. (A) GFP-labeled human B-cell acute lymphoblastic leukemia (B-ALL) cells were injected into NSG mice to create B-ALL mouse model. 21 days after B-ALL injection, aCD19 CAR-GMPs or PBS was injected and FACS analysis was performed weekly to determine the proportion of GFP-positive B-ALL cells in the peripheral blood. (B) Representative FACS analysis results. (c) Survival rates for control (PBS) and treatment (aCD19 CAR-GMPs). (D) The percentages of GFP-positive B-ALL cells in blood in control and treated mice.

### DETAILED DESCRIPTION

As used herein and in the appended claims, the singular forms “a,” “an,” and “the” include plural referents unless the context clearly dictates otherwise. Thus, for example, reference to “a cell” includes a plurality of cells and reference to “the granulocyte-macrophage progenitor” includes reference to one or more granulocyte-macrophage progenitors and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the disclosure, in connection with percentages means±1%. In other instances, as used herein, the term “about” refers to a measurable value such as an amount, a time duration, and the like, and encompasses variations of ±20%, ±10%, ±5%, ±1%, ±0.5% or ±0.1% from the specified value.

As used herein, the term “administering,” refers to the placement an agent (e.g., an engineered GMP or macrophage or granulocyte derived therefrom) as disclosed herein into a subject by a method or route which results in at least partial localization of the agents at a desired site.

“Autologous” cells as used herein refers to cells derived from the same individual as to whom the cells are later to be re-administered.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the FDA fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

A “B-Raf” kinase inhibitor refers to a substance, e.g., a compound or molecule, that blocks or reduces an activity of a protein called B-Raf kinase, or reduces an amount of B-Raf kinase. B-Raf is a kinase enzyme that helps control cell growth and signaling. It may be found in a mutated (changed) form in some types of cancer, including melanoma and colorectal cancer. Some B-Raf kinase inhibitors are used to treat cancer. Examples of B-Raf kinase inhibitor includes, but are not limited to, GDC-0879, PLX4032, GSK2118436, BMS-908662, LGX818, PLX3603, RAF265, R05185426, vemurafenib, PLX8394, and SB590885. In a particular embodiment, a method disclosed herein comprises use of the B-Raf kinase inhibitor GDC-0879.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. As non-limiting examples, “beneficial results” or “desired results” may be alleviation of one or more symptom(s), diminishment of extent of the deficit, stabilized (i.e., not worsening) state of cancer progression, delay or slowing of metastasis or invasiveness, and amelioration or palliation of symptoms associated with the cancer.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

“Chimeric antigen receptor” or “CAR” or “CARs” as used herein refers to engineered receptors, which graft an antigen specificity onto cells (for example GMP cells). CARs are also known as artificial T-cell receptors, chimeric T-cell receptors or chimeric immunoreceptors. In various embodiments, CARs are recombinant polypeptides comprising an antigen-specific domain (ASD), a hinge region (HR), a transmembrane domain (TMD), co-stimulatory domain (CSD) and an intracellular signaling domain (ISD).

“CAR binding domain” refers to the portion of the CAR that specifically binds the antigen on the target cell. In some embodiments, the binding domain of the CARs comprises any of the any of the known binding domains used in CAR constructs (see, e.g., PCT/US2017/064379) including an antibody or a functional equivalent thereof or a fragment thereof or a derivative thereof. The targeting regions may comprise full length heavy chain, Fab fragments, single chain Fv (scFv) fragments, divalent single chain antibodies or diabodies, each of which are specific to the target antigen.

“Conditions” and “disease conditions,” as used herein may include, cancers, tumors or infectious diseases. In exemplary embodiments, the conditions include, but are in no way limited to, any form of malignant neoplastic cell proliferative disorders or diseases.

A “co-stimulatory domain” as used herein refers to the portion of the CAR comprising a polypeptide domain that enhances the proliferation, survival and/or development of cells. The co-stimulatory domain is an optional domain or a CAR. The CARs of the invention may comprise no costimulatory domain or may comprise one or more co-stimulatory domains. Each co-stimulatory domain typically comprises a member of the TNFR superfamily, CD28, CD137 (4-1BB), CD134 (OX40), Dap10, CD27, CD2, CD5, ICAM-1, LFA-1(CD11a/CD18), Lck, TNFR-I, TNFR-II, Fas, CD30, CD40 or combinations thereof. Other co-stimulatory domains (e.g., from other proteins) will be apparent to those of skill in the art.

A “disease targeted by genetically modified GMPs” as used herein encompasses the targeting of any cell involved in any manner in any disease by the genetically modified GMP cells (or granulocyte or macrophage derived therefrom) of the disclosure, irrespective of whether the genetically modified cells target diseased cells or healthy cells to effectuate a therapeutically beneficial result. The genetically modified cells express the CARs, which CARs may target any of the antigens expressed on the surface of target cells. Examples of antigens which may be targeted include, but are not limited to, antigens expressed on carcinomas, sarcomas, lymphomas, leukemia, germ cell tumors, and blastomas; antigens expressed on various immune cells; and antigens expressed on cells associated with various hematologic diseases, autoimmune diseases, and/or inflammatory diseases. Other antigens that may be targeted will be apparent to those of skill in the art and may be targeted by the CARs of the disclosure.

The term “effective amount” or “therapeutically effective amount” as used herein refers to the amount of a composition comprising GMPs (macrophages or granulocytes derived therefrom) that have been engineered to express a CAR, to decrease at least one or more symptom of the disease or disorder, and relates to a sufficient amount of the composition to provide the desired effect. The phrase “therapeutically effective amount” as used herein means a sufficient amount of the composition to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the cellular compositions described herein. Measured or measurable parameters include clinically detectable markers of disease, for example, elevated or depressed levels of a biological marker. The exact amount required will vary depending on factors such as the type of disease being treated, gender, age, and weight of the subject.

An “effector function” refers to the specialized function of a differentiated cell. Effector function of a granulocyte or macrophage, for example, may be cytolytic activity or the secretion of cytokines.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses etc.) that incorporate the recombinant polynucleotide.

“Granulocyte colony-stimulating factor” or “GCSF” (also known as colony-stimulating factor 3 (CSF 3)), is a glycoprotein that stimulates the bone marrow to produce granulocytes and stem cells. The gene sequence, protein sequence and orthologs across various species are known in the art (see, e.g., NCBI Reference Sequence: NP_000750.1, which is incorporated herein by reference).

A “growth factor” refers to a substance, e.g., a compound or molecule, that is effective to promote the growth of cells, e.g., stem cells, and which, unless added to the culture medium as a supplement, is not otherwise a component of the basal medium. Growth factors include, but are not limited to, stem cell factor (SCF), basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), and vascular endothelial cell growth factor (VEGF), activin-A, Wnt and bone morphogenic proteins (BMPs), insulin, cytokines, chemokines, morphogens, neutralizing antibodies, other proteins, and small molecules. Exogenous growth factors may also be added to a medium according to the disclosure to assist in the maintenance of cultures of GMPs in a substantially undifferentiated state. Such factors and their effective concentrations can be identified as described elsewhere herein or using techniques known to those of skill in the art of culturing cells. In a particular embodiment, the GMPs are cultured in a culture medium which comprises SCF.

A “hinge region” as used herein refers to the hydrophilic region which is between the CAR binding domain and the transmembrane domain of a CAR. The hinge regions includes, but are not limited to, Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of hinge regions include, but are not limited to, CD8a hinge, and artificial spacers made of polypeptides which may be as small as, for example, Gly3 or CH1 and CH3 domains of IgGs (such as human IgG4). Other hinge regions will be apparent to those of skill in the art and may be used in connection with alternate embodiments of the invention

An “intracellular signaling domain” or “cytoplasmic domain” as used herein refers to the portion of the CAR comprising a domain that transduces the effector function signal and directs the cell to perform its specialized function. Examples of domains that transduce the effector function signal include, but are not limited to, the z chain of the T-cell receptor complex or any of its homologs (e.g., h chain, FceR1g and b chains, MB1 (Iga) chain, B29 (Igb) chain, etc.), human CD3 zeta chain, CD3 polypeptides (D, d and e), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art.

The term “isolated” as used herein refers to molecules, biologicals, cells or cellular materials being substantially free from other materials for which it is normally associated. In one aspect, the term “isolated” refers to nucleic acid, such as DNA or RNA, or protein or polypeptide (e.g., an antibody or derivative thereof), or cell or cellular organelle, separated from other DNAs or RNAs, or proteins or polypeptides, or cells or cellular organelles, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

A “linker” or “linker domain” as used herein refer to an oligo- or polypeptide region from about 1 to 100 amino acids in length, which links together any of the domains/regions of the CAR of the disclosure. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. Other linkers will be apparent to those of skill in the art.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

As used herein a “long term culture” or “long term expansion” refers to the propagation of cells under controlled conditions such that the cells expand in number and/or maintain substantial viability and substantially similar morphology. In some embodiments the term refers to the time period of culture while maintaining a desired morphology and cell number (e.g., for about two months or longer) or may be associated with the number of passages (e.g., media changes) of at least 10 media passages. In other embodiments the term refers to the increase in number over a period of time (e.g., an increase by at least one million times in a about a two-month period). In some embodiments, the long-term cultures are cultured for more than 4 months, more than 6 months or more than 1 year. In other embodiments, the long-term cultures are passaged for more than 15 passages, more than 18 passages or more than 20 passages.

“Macrophage colony-stimulating factor” or “MCSF” (also known as colony-stimulating factor 1 (CSF 1)), is involved in the proliferation, differentiation, and survival of monocytes, macrophages, and bone marrow progenitor cells. The gene sequence, protein sequence and orthologs across various species are known in the art (see, e.g., NCBI Reference Sequence: NP_000748.4, which is incorporated herein by reference).

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A typical alignment program is BLAST, using default parameters. In particular, typical programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address:
ncbi.nlm.nih.gov/cgi-bin/BLAST.

It is to be inferred without explicit recitation and unless otherwise intended, that when the present disclosure relates to a polypeptide, protein, polynucleotide, antibody or fragment thereof, an equivalent or a biologically equivalent of such is intended within the scope of this disclosure. As used herein, the term “biological equivalent thereof” is intended to be synonymous with “equivalent thereof” when referring to a reference protein, antibody or fragment thereof, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody or fragment thereof or nucleic acid. Alternatively, when referring to polynucleotides, an equivalent thereof is a polynucleotide that hybridizes under stringent conditions to the reference polynucleotide or its complement. Alternatively, when referring to polypeptides or proteins, an equivalent thereof is an expressed polypeptide or protein from a polynucleotide that hybridizes under stringent conditions to the polynucleotide or its complement that encodes the reference polypeptide or protein.

The term “retrovirus vector” refers to a vector derived from at least a portion of a retrovirus genome. Examples of retrovirus vector include MSCVneo, MSCV-pac (or MSCV-puro), MSCV-hygro as available from Addgene or Clontech. Other example of a retrovirus vector is MSCV-Bg12-AvrII-Bam-EcoR1-Xho-BstB1-Mlu-Sal-ClaI.I03 (SEQ ID NO: 872).

The term “Sleeping Beauty Transposon” or “Sleeping Beauty Transposon Vector” refers to a vector derived from at least a portion of a Sleeping Beauty Transposon genome.

“Stem Cell Factor” or “SCF” (also known as KIT-ligand, KL, or steel factor) is a cytokine that binds to the c-KIT receptor (CD117). SCF can exist both as a transmembrane protein and a soluble protein. This cytokine plays an important role in hematopoiesis (formation of blood cells), spermatogenesis, and melanogenesis. The gene sequence, protein sequence and orthologs across various species are known in the art (see, e.g., NCBI Reference Sequence NP 000890.1, which is incorporated herein by reference).

As used herein a “substantially uniform population” refers to a population of cells in which at least 80% of the cells are of the indicated type, preferably at least 90%, 95%, or even 98% or more.

A “transmembrane domain” as used herein refers to the region of the CAR which crosses the plasma membrane. The transmembrane domain of the CAR is the transmembrane region of a transmembrane protein (for example Type I transmembrane proteins), an artificial hydrophobic sequence or a combination thereof. Other transmembrane domains will be apparent to those of skill in the art. In some embodiments, the transmembrane domain can comprise transmembrane domain derived or cloned from proteins selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRFl), CD160, CD19, IL2R beta, IL2R gamma, IL7R a, ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1(CD226), SLAMF4 (CD244, 2B4), CD84, CD96 (Tactile), CEACAMI1, CRT AM, Ly9 (CD229), CD160 (BY55), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKp44, NKp30, NKp46, NKG2D, and/or NKG2C.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

A “Wnt activator” refers to compound or molecule that induces Wnt signaling pathways. The Wnt signaling pathways are a group of signal transduction pathways which begin with proteins that pass signals into a cell through cell surface receptors. Three Wnt signaling pathways have been characterized: the canonical Wnt pathway, the noncanonical planar cell polarity pathway, and the noncanonical Wnt/calcium pathway. All three pathways are activated by the binding of a Wnt-protein ligand to a Frizzled family receptor, which passes the biological signal to the Disheveled protein inside the cell. Wnt comprises a diverse family of secreted lipid-modified signaling glycoproteins that are 350-400 amino acids in length. The type of lipid modification that occurs on these proteins is palmitoylation of cysteines in a conserved pattern of 23-24 cysteine residues. Palmitoylation is necessary because it initiates targeting of the Wnt protein to the plasma membrane for secretion and it allows the Wnt protein to bind its receptor due to the covalent attachment of fatty acids. Wnt proteins also undergo glycosylation, which attaches a carbohydrate in order to ensure proper secretion. In Wnt signaling, these proteins act as ligands to activate the different Wnt pathways via paracrine and autocrine routes. These proteins are highly conserved across species. They can be found in mice, humans, Xenopus, zebrafish, Drosophila and many others. Examples of Wnt activators includes, but are not limited to, SKL 2001, BML-284, WAY 262611, CAS 853220-52-7, and QS11. In a particular embodiment, a method disclosed herein comprises use of a compound of the disclosure which has Wnt activator activity.

Granulocytes and macrophages are the two major cell types of the innate immune system. They are the first line of defense against pathogens and also play a central role in maintaining the homeostasis of our bodies and preventing infections and various diseases, including metabolic diseases and cancers. Granulocytes and macrophages engulf and digest invading microorganisms in a process called phagocytosis. Besides phagocytosis, macrophages also play a critical role as antigen presenters, initiating specific defense mechanisms (adaptive immunity) by recruiting other immune cells such as lymphocytes. Recently, macrophages have also become an attractive therapeutic target to combat cancer. Despite their huge therapeutic potential, there is no effective method to expand and genetically modify granulocytes and macrophages, greatly limiting their clinical application.

A more promising approach is to expand and genetically modify the granulocyte-monocyte progenitor (GMP) cells, which is the common progenitor in the bone marrow from which granulocytes and macrophages originate. As used herein, the GMPs can be derived from or obtained from a mammalian species (e.g., bovine, canine, equine, feline, human, murine, primate, rat etc.). Ex vivo expanded GMPs could allow the ability to generate ample macrophages for therapeutic applications. Expanded GMPs could also be induced to differentiate into the most abundant type of granulocyte, the neutrophil, which could then be infused into blood circulation to fight infection in patients with neutropenia or neutrophil dysfunction. More importantly, GMPs could easily be modified to generate genetically engineered macrophages with enhanced antitumor or antimicrobial activity. Macrophages engulf and digest any foreign particles including the genetic materials used to engineer them, but GMPs do not have phagocytic activity, making them a much more favorable target. However, despite decades of intensive studies, the long-term ex vivo expansion of GMPs, as well as other stem/progenitor cells of the hematopoietic system, is yet to be realized.

The cells of the hematopoietic system are organized in a hierarchy with hematopoietic stem cells (HSCs) at the top, various mature blood cells at the bottom, and intermediate hematopoietic stem and progenitor cells (HSPCs) such as GMPs in between. Lured by the great therapeutic potential of HSPCs, numerous groups have attempted to develop culture conditions for their ex vivo expansion in the past two decades. So far, all the conditions have a fundamental limitation: they are unable to continuously and exponentially expand homogeneous populations of any type of HSPC.

A unique challenge has been the difficulty of distinguishing and separating one type of HSPC from another, and particularly from its immediate upstream progenitors and downstream progeny. In fact, the conventional immunophenotypic analysis cannot distinguish one HSPC type from its immediate upstream progenitors and downstream progeny. At the clonal level, prospectively purified HSCs are still highly heterogeneous, containing cells with diverse gene expression patterns and distinct cellular functions. This is expected, because HSPCs span a continuum of cells with somewhat similar cell surface marker expression, but heterogeneous functions. Within this heterogenous cell population, it is technically very challenging to identify growth factors/cytokines and small molecules that can promote the expansion of a single stem/progenitor cell type.

Granulocytes, macrophages, and dendritic cells originate from a common progenitor in the bone marrow, the granulocyte-macrophage progenitor (GMP). Despite the immense therapeutic potential of innate immune cells, their application in the clinic has been greatly limited because of the current inability to effectively expand and genetically modify these cells or their progenitors GMPs. Provided herein are methods for the long-term expansion of GMPs. Ex vivo expanded GMPs can efficiently differentiate into mature and functional granulocytes, macrophages, and dendritic cells both in vitro and in vivo. These ex vivo expanded GMPs can also be genetically modified. The methods disclosed herein for the production of GMPs, and the GMPs produced therefrom, have great utility because: (1) long-term expansion of GMPs provide unlimited homogenous cell populations for both basic research and clinical applications; (2) long-term expansion of GMPs allows for the studying the regulation of an immune response by modifying GMP genes, and their expression thereof; and (3) ex vivo expanded GMPs can be used for clinical applications, including transplantation. For example, ex vivo expanded GMPs can readily be used to treat neutropenia. Moreover, the disclosure also provides for the genetic modification of GMPs (e.g., knockout SIRPα and/or PI3Kγ gene; overexpression of angiotensin converting enzyme), which can be further induced to differentiate into macrophages and dendritic cells. In the studies presented herein these engineered macrophages and dendritic cells exhibit enhanced antitumor effects and can be used clinically to treat cancer, either as monotherapy or combination therapy with other immunological agents, such as anti-PD-1/PD-L1 antibodies and chimeric antigen receptor T (CAR-T) cells. GMPs were also engineered to produce CAR-macrophages. These CAR-macrophages can be used treat cancer and other diseases.

Macrophages display divergent phenotypes that were originally classified as M1 or M2 polarity. M1 polarized macrophages display the capacity to present antigen, produce IL-12, IL-23, interferon gamma (IFNγ), and reactive oxygen species (ROS). M1 macrophages are more effective at antitumor and skewing T cell responses toward a T helper type 1 (Th1) or cell mediated immune response. In contrast, M2 macrophages produce IL-10 and TGF-β and participate in tissue remodeling, have immunosuppressive qualities, and promote Th2 or antibody mediated immune responses. Tumor-associated macrophages (TAMs) constitute a major component of the tumor microenvironment. These cells are predominant M2 phenotype macrophages which promote tumor immunosuppression. Recent studies support their contribution to the suppression of T cell function, which is not abolished by the use of Immune checkpoint blockage. Macrophages have therefore become an attractive therapeutic target to combat cancer. Despite the huge therapeutic potential of macrophages, their application in clinic has been greatly limited because currently there is no effective method to expand and genetically modify macrophages or their progenitors GMPs. Long-term expansion of GMPs allows for genetic modification to make these cells more therapeutically applicable.

In a particular embodiment, the disclosure provides a method for the long-term expansion of a uniform cell population of granulocyte/macrophage progenitor cells (GMPs) that remain morphologically unchanged after undergoing multiple cell passages and clonal expansion. In a further embodiment, a method disclosed herein comprises the step of culturing GMPs in a culture medium which includes a combination of factors and agents including, but not limited to, a growth factor (e.g., SCF), a B-Raf kinase inhibitor (e.g., GDC-0879), an agent that inhibits Mnk1/2, an agent that inhibits the PI3K pathway, and optionally, one or more serum components. In still another embodiment, the long-term culture of the GMPs are genetically engineered to express a chimeric antigen receptor (CAR).

Stem cells are cells capable of differentiation into other cell types, including those having a particular, specialized function (e.g., tissue specific cells, parenchymal cells and progenitors thereof). Progenitor cells (i.e., “multipotent”) are cells that can give rise to different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. As will be appreciated, “multipotent” stem/progenitor cells (e.g., granulocyte/macrophage progenitor cells (GMPs)) have a narrower differentiation potential than do pluripotent stem cells. Prior to derivation into GMPs, the stem cells disclosed herein can be genetically modified by use of any number of genetic engineering techniques, e.g., such as gene therapy, gene editing systems, homologous recombination, etc. Such modified stem cells may provide for enhanced therapies (e.g., see Nowakowski et al., Acta Neurobiol Exp (Wars) 73(1):1-18 (2013)). In certain embodiments, a stem cell or progenitor cell may be engineered to express, or contain a polynucleotide encoding, a chimeric antigen receptor (CAR).

In a further embodiment, the GMPs disclosed herein are derived from stem cells. Stem cells can include embryonic stem cells, induced pluripotent stem cells, non-embryonic (adult) stem cells, and cord blood stem cells. Stem cell types that can be cultured using the media of the disclosure include stem cells derived from any mammalian species including humans, mice, rats, monkeys, and apes (see, e.g., Nature 448:313-318, July 2007; and Takahashi et al., Cell 131(5):861-872; which are incorporated herein by reference).

In a particular embodiment, the GMPs of the disclosure are derived from induced pluripotent stem cells (iPSs, or iPSCs). iPSCs are a type of pluripotent stem cell obtained from non-pluripotent cells by selective gene expression (of endogenous genes) or by transfection with a heterologous gene. Induced pluripotent stem cells are described by Shinya Yamanaka's team at Kyoto University, Japan. Yamanaka et al. had identified genes that are particularly active in embryonic stem cells, and used retroviruses to transfect mouse fibroblasts with a selection of those genes. Eventually, four key pluripotency genes essential for the production of pluripotent stem cells were isolated: Oct-3/4, SOX2, c-Myc, and Klf4. More recent research has provided the fewer of these factors in combination with certain culture conditions as well as additional factors can induce pluripotent stem cells. Cells were isolated by antibiotic selection for Fbx15⁺ cells. The same group published a study along with two other independent research groups from Harvard, MIT, and the University of California, Los Angeles, showing successful reprogramming of mouse fibroblasts into iPS and even producing a viable chimera.

In an alternate embodiment, the GMPs disclosed herein are derived from embryonic stem cells (ESCs). ESCs are stem cells derived from the undifferentiated inner mass cells of a human embryo. Embryonic stem cells are pluripotent, meaning they are able to grow (i.e. differentiate) into all derivatives of the three primary germ layers: ectoderm, endoderm and mesoderm. Pluripotency distinguishes embryonic stem cells from adult stem cells found in adults; while embryonic stem cells can generate all cell types in the body, adult stem cells are multipotent and can produce only a limited number of cell types. Additionally, under defined conditions, embryonic stem cells are capable of propagating themselves indefinitely. This allows embryonic stem cells to be employed as useful tools for both research and regenerative medicine, because they can produce limitless numbers of themselves for continued research or clinical use.

In another alternate embodiment, the GMPs disclosed herein are derived from cord blood stem cells. Umbilical cord blood is the blood left over in the placenta and in the umbilical cord after the birth of the baby. The cord blood is composed of all the elements found in whole blood. It contains red blood cells, white blood cells, plasma, platelets and is also rich in hematopoietic stem cells. Hematopoietic stem cells can be isolated from cord blood using any number of isolation methods taught in the art, including those taught in Chularojmontri et al., J Med Assoc Thai 92(3):S88-94 (2009). Moreover, commercial kits are available for isolation CD34⁺ cells (i.e., hematopoietic stem cells) from human umbilical cord blood from multiple vendors, including STEMCELL Technologies, Thermo Fisher Scientific, Zen-Bio, etc.

In yet another alternate embodiment, the GMPs disclosed herein are derived from non-embryonic stem cells. The non-embryonic stem cell can renew itself and can differentiate to yield some or all of the major specialized cell types of the tissue or organ. The primary roles of non-embryonic stem cells in a living organism are to maintain and repair the tissue in which they are found. Scientists also use the term somatic stem cell instead of non-embryonic stem cell, where somatic refers to cells of the body (not the germ cells, sperm or eggs). Non-embryonic stem cells have been identified in many organs and tissues, including brain, bone marrow, peripheral blood, blood vessels, skeletal muscle, skin, teeth, heart, gut, liver, ovarian epithelium, and testis. They are thought to reside in a specific area of each tissue (called a “stem cell niche”). In a living animal, non-embryonic stem cells are available to divide for a long period, when needed, and can give rise to mature cell types that have characteristic shapes and specialized structures and functions of a particular tissue.

In a particular embodiment, the GMPs disclosed herein are derived from hematopoietic stem cells (HSCs). HSCs can easily be isolated from umbilical cord blood and bone marrow. Such isolation protocols are known in the art and typically use CD34⁺ as a cell selection marker for the isolation of HSCs (e.g., see Lagasse et al., Nat Med. 6:1229-1234(2000)).

In the methods disclosed herein, the GMPs can be grown and expanded in a culture medium which includes a combination of factors and agents including, but not limited to, a growth factor (e.g., SCF), a B-Raf kinase inhibitor (e.g., GDC-0879), an agent that inhibits Mnk1/2, an agent that inhibits the PI3K pathway, and optionally, one or more serum components. The culture medium can be a modified basal medium that is supplemented with various other biological agents. A basal medium refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cell's survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of basal media are known in the art of mammalian cell culture, such as Dulbecco's Modified Eagle Media (DMEM), RPMI 1640, Knockout-DMEM (KO-DMEM), and DMEM/F12, although any base medium that can be supplemented with agents which supports the growth of stem cells in a substantially undifferentiated state can be employed. It was further found herein, that a culture medium that comprises a ratio of one of the basal medias exemplified above (e.g., DMEM/F12) with a neural basal medium (or alternatively other basal medium such as IMDM and/or StemSpan™ SFEMII) unexpectedly provided for improved growth of the GMPs. In particular, a ratio of about 5:1 to about 1:5 of one of the basal medias exemplified above (e.g., DMEM/F12) to a neural basal medium can be used to culture the GMPs. In a further embodiment, the culture medium for growing GMPs comprises about 1:1 of DMEM/F12 to a neural basal media.

As indicated above, the culture medium disclosed herein for growing GMPs may be supplemented with one or more additional agents, including, but not limited to insulin, transferrin, BSA fraction V, putrescine, sodium selenite, DL-α tocopherol, and linolenic acid. In a certain embodiment, the culture medium disclosed herein for growing GMPs is supplemented with insulin, transferrin, BSA fraction V, putrescine, sodium selenite, DL-α tocopherol, and linolenic acid.

As will be appreciated, it is desirable to replace spent culture medium with fresh culture medium either continually, or at periodic intervals, typically every 1 to 3 days. One advantage of using fresh medium is the ability to adjust conditions so that the cells expand more uniformly and rapidly than they do when cultured on feeder cells according to conventional techniques, or in conditioned medium.

Populations of GMPs can be obtained that are 4-, 10-, 20-, 50-, 100-, 1000-, or more fold expanded when compared to the previous starting cell population. Under suitable conditions, cells in the expanded population will be 50%, 70%, or more in the undifferentiated state, as compared to the GMPs used to initiate the culture. The degree of expansion per passage can be calculated by dividing the approximate number of cells harvested at the end of the culture by the approximate number of cells originally seeded into the culture. Where geometry of the growth environment is limiting or for other reasons, the cells may optionally be passaged into a similar growth environment for further expansion. The total expansion is the product of all the expansions in each of the passages. Of course, it is not necessary to retain all the expanded cells on each passage. For example, if the cells expand two-fold in each culture, but only about 50% of the cells are retained on each passage, then approximately the same number of cells will be carried forward. But after four cultures, the cells are said to have undergone an expansion of 16-fold. Cells may be stored by cryogenic freezing techniques known in the art.

As indicated in more detail herein, the GMPs can be grown and expanded in a culture medium which includes a combination of factors and agents including, but not limited to, a growth factor (e.g., SCF), a B-Raf kinase inhibitor (e.g., GDC-0879), an agent that inhibits Mnk1/2, an agent that inhibits the PI3K pathway, and optionally, one or more serum components.

The disclosure provides methods to genetically modify the GMPs disclosed herein using genetic engineering techniques. In particular it was shown herein that the GMPs of the disclosure are susceptible to genetic modification techniques, thereby allowing for the use of the GMPs in basic scientific research and clinical therapeutic applications. Thus, expanded and genetically modified GMPs can be readily translated into broad clinical applications. Accordingly, the disclosure further provides methods to genetically modify GMPs disclosed herein. Such methods, can include the step of genetically engineering modifications into GMPs by using a gene editing system, homologous recombination, or site directed mutagenesis. Particular examples of gene editing systems include zing finger nucleases, TALEN and CRISPR.

In a certain embodiment, the CRISPR system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archaea, Mole Cell 2010, January 15; 37(1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to target sequences comprising the protospacer and the corresponding PAM via heteroduplex formation between the spacer region of the crRNA and the protospacer DNA. Finally, Cas9 mediates cleavage of target sequence of PAM to create a DSB within the protospacer. In a certain embodiment, the RNA polymerase Ill-based U6 promoter is to drive the expression of tracrRNA.

Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell (e.g., a GMP or stem cell) such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequences, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a CRISPR expression vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell (e.g., a GMP or stem cell). In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools. In particular, any or all of the following mutations are preferred in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s are also indicated.

Indicated orthologs are also described herein. A Cas enzyme may be identified Cas9 as this can refer to the general class of enzymes that share homology to the biggest nuclease with multiple nuclease domains from the type II CRISPR system. Most preferably, the Cas9 enzyme is from, or is derived from, spCas9 or saCas9. By derived, it is meant that the derived enzyme is largely based, in the sense of having a high degree of sequence homology with, a wildtype enzyme, but that it has been mutated (modified) in some way as described herein.

It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure includes many more Cas9s from other species of microbes, such as SpCas9, SaCa9, St1Cas9 and so forth.

The gene editing systems (e.g., zing finger nucleases, CRISPR and TALEN) can be used to genetically engineer modifications into the GMP or stem cells, such as replacing or disrupting an existing gene found in the GMP or stem cell (knockout). As shown in the Examples presented herein, the GMPs of the disclosure are particular susceptible to knockout mutations. Moreover, it is expected that additional knockouts could be easily created from the GMPs of the disclosure such as SIRPα gene knockouts and/or a PI3Kγ gene knockouts. Alternatively, the same editing systems (e.g., CRISPR and TALEN) can be used to alter a genetic locus to contain sequence information not found at the genetic locus (a knock-in mutation). Such modifications, can be used to create GMP's that have “gained a function.” Such modified GMPs are particular useful for mimicking a disease state, e.g., by expressing biomolecules associated with a disease or disorder.

In another embodiment, the GMP cells are engineered using a vector. For example, a CAR of the disclosure can be introduced into a cell using any number of techniques including, but not limited to, using lentiviral vectors, retroviral vectors, adeno-associated viral vectors, baculovirus vectors, sleeping beauty transposons, piggybac transposons or by mRNA transfection, or using a combination of the above methods. The CAR can be expressed so that they are under the control of an endogenous promoter (e.g., TCRα or TCRβ promoter). In some embodiments, a CAR is expressed using foreign promoters (e.g. a CMV promoter).

In some embodiment, the introducing the nucleic acid molecule encoding a CAR comprises transducing a vector comprising the nucleic acid molecule encoding a CAR, or transfecting the nucleic acid molecule encoding a CAR, into GMPs cultured as described herein.

In some embodiments, the method comprises: a) providing a population of GMPs cultured to expand and maintain the culture of GMPs; b) introducing a vector comprising a nucleic acid encoding a CAR construct into the GMPs; and c) culturing the transformed/transfected GMPs. In some embodiments, the method further provides for the differentiation of the GMPs into myeloid and lymphoid lineages of blood cells, such as monocytes, macrophages, granulocytes, neutrophils, basophils, eosinophils, erythrocytes, megakaryocytes to platelets, T cells, B cells, and natural killer cells. In a particular embodiment, a method disclosed herein further comprises differentiating the GMPs of the disclosure into macrophages by culturing the GMPs with a macrophage differentiation medium comprising MCSF. In yet a further embodiment, the macrophage differentiation medium comprises RPMI 1640, 10% FBS and 20 ng/mL of MCSF. In an alternate embodiment, a method disclosed herein further comprises differentiating the GMPs of the disclosure into granulocytes comprising: culturing the GMPs with a granulocyte differentiation medium comprising GCSF. In yet a further embodiment, the granulocyte differentiation medium comprises RPMI 1640, 10% FBS and 20 ng/mL of GCSF.

In a particular embodiment, the disclosure provides a method to genetically engineer granulocyte-macrophage progenitors (GMPs) to express a chimeric antigen receptor (CAR) comprising: introducing a vector comprising a CAR into GMPs to form GMPs that express CAR (CAR-GMPs); expanding and culturing the CAR-GMPs for multiple passages in defined culture conditions to generate a population of CAR-GMPs; and inducing the population of CAR-GMPs to differentiate into granulocytes, macrophages or dendritic cells in vitro, wherein the granulocytes, macrophages or dendritic cells express CAR. In a further embodiment, the GMPs are obtained from stem cells. In yet a further embodiment, the stem cells are hematopoietic stem cells. In another embodiment, the hematopoietic stem cells are isolated from the bone marrow of a subject. In yet another the subject is a mammalian subject. In a further embodiment, the subject is a human patient. In yet a further embodiment, the CAR comprises an extracellular domain capable of binding to an antigen, a transmembrane domain and at least one intracellular domain that is designed to increase the anti-tumor activities of granulocytes, macrophages and dendritic cells by increasing their phagocytosis and/or proinflammatory cytokines secretion. In another embodiment, the vector is a viral vector. In yet another embodiment, the viral vector can be replicating or non-replicating, and can be an adenoviral vector, an adeno-associated virus (AAV) vector, a measles vector, a herpes vector, a retroviral vector, a lentiviral vector, a rhabdoviral vector, a reovirus vector, a Seneca Valley Virus vector, a poxvirus vector, a parvovirus vector, or an alphavirus vector. In a certain embodiment, the viral vector is a lentiviral vector. In another embodiment, the defined culture conditions include culturing the CAR-GMPs in a culture medium comprising: (i) a growth factor, (ii) a B-Raf kinase inhibitor, and (iii) a Wnt activator and/or a GSK-3 inhibitor, wherein the CAR-GMPs remain substantially morphologically unchanged after undergoing multiple cell passages and/or clonal expansion. In a further embodiment, the culture medium comprises DMEM/F12 and Neural Basal Medium. In yet a further embodiment, the culture medium comprises DMEM/F12 and Neural Basal Medium in a ratio of about 5:1 to about 1:5. In another embodiment, the culture medium comprises DMEM/F12 and Neural Basal Medium in a ratio of about 1:1. In yet another embodiment, the culture medium comprises one or more supplements selected from insulin, transferrin, bovine serum albumin (BSA) fraction V, putrescine, sodium selenite, DL-α tocopherol, and/or linolenic acid. In a further embodiment, the culture medium is supplemented with insulin, transferrin, BSA fraction V, putrescine, sodium selenite, DL-α tocopherol, and linolenic acid. In a certain embodiment, the growth factor is stem cell factor (SCF). In another embodiment, the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof. In yet another embodiment, the Wnt activator is selected from the group consisting of SKL 2001, BML-284, WAY 262611, CAS 853220-52-7, QS11 and any combination thereof. In a further embodiment, the GSK-3 inhibitor is selected from the group consisting of CHIR99021, CHIR98014, SB216763, BIO, A1070722, AR-A014418 and any combination thereof. In another embodiment, the defined culture conditions include culturing the CAR-GMPs in a culture medium comprising: (i) a growth factor; (ii) a B-Raf kinase inhibitor; (iii) an agent that inhibits the mitogen-activated kinase interacting protein kinases 1 and 2 (Mnk1/2); (iv) an agent that inhibits the PI3K pathway; (v) optionally, one or more serum components; wherein the CAR-GMPs remain substantially morphologically unchanged after undergoing multiple cell passages and/or clonal expansion. In a further embodiment, the culture medium comprises DMEM/F12 and Neural Basal Medium. In yet a further embodiment, the culture medium comprises DMEM/F12 and Neural Basal Medium in a ratio of about 5:1 to about 1:5. In another embodiment, the culture medium comprises DMEM/F12 and Neural Basal Medium in a ratio of about 1:1. In yet another embodiment, the culture medium comprises one or more supplements selected from insulin, transferrin, bovine serum albumin (BSA) fraction V, putrescine, sodium selenite, DL-α tocopherol, and/or linolenic acid. In a further embodiment, the culture medium is supplemented with insulin, transferrin, BSA fraction V, putrescine, sodium selenite, DL-α tocopherol, and linolenic acid. In yet a further embodiment, the growth factor is stem cell factor (SCF). In another embodiment, the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof.

In a further embodiment, the agent that inhibits Mnk1/2 is selected from the group consisting of CGP-57380, cercosporamide, BAY 1143269, tomivosertib, ETC-206, SLV-2436 and any combination thereof. In yet a further embodiment, the agent that inhibits PI3K pathway is selected from the group consisting of 3-methyladenine, LY294002, alpelisib, wortmannin, quercetin, hSMG-1 inhibitor 11j, zandelisib, alpelisib hydrochloride, idelalisib, buparlisib, copanlisib, IPI549, dactolisib, pictilisib, SAR405, duvelisib, fimepinostat, GDC-0077, PI-103, YM-20163, PF-04691502, Taselisib, omipalisib, samotolisib, isorhamnetin, ZATK474, parsaclisib, rigosertib, AZD8186, GSK2636771, disitertide, TG100-115, AS-605240, PI3K-IN-1, dactolisib tosylate, gedatolisib, TGX-221, umbralisib, AZD 6482, serabelisib, bimiralisib, apitolisib, alpha-linolenic acid, Vps34-PIK-III, PIK-93, Vps34-IN-1, CH5132799, leniolisib, voxtalisib, GSK1059615, sonolisib, PKI-402, PI4KIIIbeta-IN-9, HS-173, BGT226 maleate, pictilisib dimethane sulfonate, VS-5584, IC-87114, quercetin dihydrate, CNX-1351, SF2523, GDC-0326, seletalisib, acalisib, SAR-260301, ZAD-8835, GNE-317, AMG319, nemiralisib, IITZ-01, PI-103 hydrochloride, oroxin B, pilaralisib, AS-252424, cpanlisib dihydrochloride, AMG 511, disitertide TFA, PIK-90, tenalisib, esculetin, CGS 15943, GNE-477, PI-3065, A66, AZD3458, ginsenoside Rk1, sophocarpine, buparlisib hydrochloride, Vps34-IN-2, linperlisib, arnicolide D, KP372-1, CZC24832, PF-4989216, (R)-Duvelisib, PQR530, P11δ-IN-1, umbralisib hydrochloride, MTX-211, PI3K/mTOR Inhibitor-2, LX2343, PF-04979064, polygalasaponin F, glaucocalyxin A, NSC781406, MSC2360844, CAY10505, IPI-3063, TG 100713, BEBT-908, PI-828, brevianamide F, ETP-46321, PIK-294, SRX3207, sophocarpine monohydrate, AS-604850, desmethylglycitein, SKI V, WYE-687, NVP-QAV-572, GNE-493, CAL-130 hydrochloride, GS-9901, BGT226, IHMT-PI3Kδ-372, PI3Kα-IN-4, parsaclisib hydrochloride, PF-06843195, PI3K-IN-6, (S)-PI3Kα-IN-4, PI3K(gamma)-IN-8, BAY1082439, CYH33, PI3Kγ inhibitor 2, PI3Kδ inhibitor 1, PARP/PI3K-IN-1, LAS191954, PI3K-IN-9, CHMFL-PI3KD-317, PI3K/HDAC-IN-1, MSC2360844 hemifumarate, PI3K-IN-2, PI3K/mTOR Inhibitor-1, PI3Kδ-IN-1, euscaphic acid, KU-0060648, AZD 6482, WYE-687 dihydrochloride, GSK2292767, (R)-Umbralisib, PIK-293, idelalisib D5, PIK-75, hirsutenone, quercetin D5, PIK-108, hSMG-1 inhibitor 11e, PI3K-IN-10, NVP-BAG956, PI3Kγ inhibitor 1, CAL-130, ON 146040, PI3kδ inhibitor 1, PI3Kα/mTOR-IN-1, and any combination thereof. In another embodiment, the CAR-GMPs are induced to differentiate into macrophages comprising: culturing the CAR-GMPs with a macrophage differentiation medium comprising macrophage colony-stimulating factor (MCSF), wherein the macrophages express CAR. In yet another embodiment, the macrophage differentiation medium comprises RPMI 1640, fetal bovine serum (FBS) and MCSF. In an alternate embodiment, the method further comprises differentiating the CAR-GMPs into granulocytes comprising: culturing the GMPs with a granulocyte differentiation medium comprising granulocyte colony-stimulating factor (GCSF), wherein the granulocytes express CAR. In a further embodiment, the granulocyte differentiation medium comprises RPMI 1640, FBS and GCSF.

In the studies presented herein, it was found that ex vivo expanded GMPs can be engineered to produce CAR-macrophages that target cancer cells with high efficiency and specificity. Accordingly, the disclosure provides methods to genetically engineer granulocyte-macrophage progenitors (GMPs) to express a chimeric antigen receptor (CAR) for the use of cancer immunotherapy. The chimeric antigen receptor comprises an extracellular domain capable of binding to an antigen, a transmembrane domain and at least one intracellular domain. The intracellular domain is designed to increase the anti-tumor activities of granulocytes, macrophages and dendritic cells by increasing their phagocytosis and/or proinflammatory cytokines secretion. The CAR-GMPs are expanded and can be induced to differentiate into granulocytes, macrophages or dendritic cells in vitro or in vivo. The CAR-GMPs or their derivatives granulocytes, macrophages, and dendritic cells are adoptively transferred into patients where they act as a potent immune effector by infiltrating the tumor and killing the target cells.

The CAR-GMPs can be further administered in combination with one or more anticancer agents to treat a subject with cancer. Examples, of anticancer agents that can be used with the CAR-GMPs disclosed herein include, but are not limited to, alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above.

There are several advantages of this GMP-based cancer immunotherapy over other types of cellular immunotherapies, such as CAR-T therapy. Long-term expansion of GMPs offers the opportunity for producing off-the-shelf CAR-macrophages for immunotherapy. A major hurdle in the clinical application of off-the-shelf CAR-macrophages is human leukocyte antigen (HLA) compatibility. The ability to long-term expand and genetically engineer GMPs allows the establishment of a master cell bank of GMPs collected from healthy donors and/or umbilical cord blood for generating off-the-shelf CAR-macrophages for immunotherapy. Alternatively, HLA-universal GMPs can be generated using gene modification techniques as described above. Long-term expansion of GMPs allows for sophisticated and multiplex genetic engineering on GMPs to render these cells more therapeutically applicable. For example, the signal-regulatory protein-α (SIRPα) and phosphatidylinositol 3-kinase-γ (PI3Kγ) genes can be knocked out to further enhance the antitumor activity of GMP-derived CAR-macrophages. SIRPα knockout in macrophages is expected to enhance their antitumor activity by disrupting the CD47-SIRPα interaction between tumor cells and macrophages. PI3Kγ is abundantly expressed in macrophages and directly controls a macrophage switch between immune stimulation (M1 macrophage) and suppression (M2 macrophage). Activation of PI3Kγ in macrophages induces a transcriptional program that promotes immune suppression during inflammation and tumor growth, whereas inactivation of macrophage PI3Kγ promotes an immunostimulatory transcriptional program. It is expected that PI3Kγ−/− macrophages will have enhanced antitumor activity by polarizing to an immune stimulatory M1 phenotype.

Adoptive transfer of genetically engineered GMPs has the potential to reverse the immunosuppressive tumor microenvironment (TME). Tumor-associated macrophages (TAMs) constitute a major component of the TME. Experimental and clinical studies have found that the majority of TAMs are immunosuppressive M2 macrophages that prevent tumor cells from being attacked by natural killer (NK) and T cells. These observations suggest a need to target TAMs in combination with other immunotherapies in order to achieve maximal antitumor effect. One strategy is to replenish immunosuppressive M2 macrophages with immunostimulatory M1 macrophages that have antitumor activity. This can be achieved by depleting TAMs followed by adoptive transfer of immunostimulatory M1 macrophages generated from PI3Kγ^{-/-} GMPs or GMPs overexpressing IL-12. Monocytes and macrophages expressing IL-12 have been shown to change the TME from immunosuppressive to immunostimulatory.

GMPs can be engineered to produce macrophages with the potential to mount more complete and robust immune responses than CAR-T cells. Macrophages exhibit their antitumor activity through the secretion of inflammatory cytokines, the phagocytosis of cancer cells, and more importantly, the processing and presentation of cancer antigens to NK and T cells. Macrophages are professional antigen-presenting cells (APCs). Endogenous NK and T cells activated by macrophages are likely to mount an immune response with high selectivity and efficiency. Therefore, harnessing the power of GMPs/macrophages through genetic engineering represents a promising approach for developing the next-generation cancer immunotherapy.

The disclosure provides a method of treating or preventing a disease associated with expression of a disease-associated antigen in a subject, comprising administering to the subject an effective amount of an GMP (or macrophage, granulocyte etc. derived therefrom) comprising a chimeric antigen receptor (CAR), wherein the CAR comprises an antigen binding domains that bind to the disease-associated antigen associated with the disease, and said disease-associated antigen is selected from a group consisting of: CD5, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(l-4)bDGlcp(l-l)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-llRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(l-4)bDGlcp(l-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member lA (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin Bl; v-myc avian myelocytomatosis viral oncogene neuroblastoma derived homolog (MYCN); Ras Homolog Family Member C (RhoC); Tyrosinase-related protein 2 (TRP-2); Cytochrome P450 lB 1 (CYPlB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUl); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGLl1, ALK TCR gamma-delta, NKG2D, CD32 (FCGR2A), CSPG4-HMW-MAA, Timl-/HVCR1, CSF2RA (GM-CSFR-alpha), TGFbetaR2, VEGFR2/KDR, Lewis Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC),KSHV-K8.1 protein, KSHV-gH protein, auto antibody to desmoglein 3 (Dsg3), autoantibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin18.2 (CLD18A2 OR CLDN18A.2)), P-glycoprotein, STEAPI, LIV1, NECTIN-4, CRIPTO, GPA33, BST1/CD157, low conductance chloride channel, and antigen recognized by TNT antibody, thereby treating the subject or preventing a disease in the subject.

In another aspect, a method of treating a subject comprises administering an effective amount of a GMP (or macrophage, granulocyte etc. derived therefrom) comprising a chimeric antigen receptor (CAR) for reducing or ameliorating a hyperproliferative disorder or condition (e.g., a cancer), e.g., solid tumor, a soft tissue tumor, a blood cancer, or a metastatic lesion, in a subject is provided. As used herein, the term “cancer” is meant to include all types of cancerous growths or oncogenic processes, metastatic tissues or malignantly transformed cells, tissues, or organs, irrespective of histopathologic type or stage of invasiveness. Exemplary solid tumors include malignancies, e.g., adenocarcinomas, sarcomas, and carcinomas, of the various organ systems, such as those affecting breast, liver, lung, brain, lymphoid, gastrointestinal (e.g., colon), genitourinary tract (e.g., renal, urothelial cells), prostate and pharynx. Adenocarcinomas include cancers such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the disclosure. Examples of other cancers that can be treated or prevented include pancreatic cancer, bone cancer, skin cancer, cutaneous or intraocular malignant melanoma, uterine cancer, ovarian cancer, rectal cancer, cancer of the head or neck, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In another aspect, a method of treating a subject comprises administering an effective amount of a GMP (or macrophage, granulocyte etc. derived therefrom) comprising a chimeric antigen receptor (CAR) for reducing or ameliorating a hyperproliferative disorder or condition (e.g., a cancer), e.g., solid tumor, a soft tissue tumor, a blood cancer, or a metastatic lesion, in a subject is provided. As used herein, the term “cancer” is meant to include all types of cancerous growths or oncogenic processes, metastatic tissues or malignantly transformed cells, tissues, or organs, irrespective of histopathologic type or stage of invasiveness. Exemplary solid tumors include malignancies, e.g., adenocarcinomas, sarcomas, and carcinomas, of the various organ systems, such as those affecting breast, liver, lung, brain, lymphoid, gastrointestinal (e.g., colon), genitourinary tract (e.g., renal, urothelial cells), prostate and pharynx. Adenocarcinomas include cancers such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the disclosure. Examples of other cancers that can be treated or prevented include pancreatic cancer, bone cancer, skin cancer, cutaneous or intraocular malignant melanoma, uterine cancer, ovarian cancer, rectal cancer, cancer of the head or neck, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

The following examples are intended to illustrate but not limit the disclosure. While they are typical of those that might be used, other procedures known to those skilled in the art may alternatively be used.

### Examples

Mice. C57BL/6J (JAX Stock #000664), B6.129(Cg)-Gt(ROSA)26Sortm4(ACTB-tdTomato,-EGFP)Luo/J (mTmG, JAX stock #007676), B6.129S-Cybbtm1Din/J (gp91phox-, JAX Stock #002365), NOD.Cg-Prkdcscid Il2rgtm1Wjl (NSG, JAX stock #05557) and B6J.129(Cg)-Gt(ROSA)26Sortm1.1(CAG-cas9*,-EGFP)Fezh/J (CAG-Cas9-EGFP, JAX stock #026179) mice were purchased from Jackson Laboratory. All mice (male and female) were used between the age of 6 and 12 weeks old. All animal experiments were performed in accordance with protocols approved by the University of Southern California Animal Care and Use Committee. Animals (5 mice per cage) were provided food and water and were maintained on a regular 12-h light-dark cycle. NSG mice were bred under sterile condition

CGD mouse model. gp91phox-mice (CGD mice) were irradiated with a lethal dose (950 cGy) and transplanted with either 5×10⁶ tdTomato-positive GMPs and 2.5×10⁴ gp91phox-whole bone marrow cells (helper cells) or 2.5×10⁴ helper cells only via tail vein injection. Two days after transplantation, mice were injected intraperitoneally with 2×10⁸ S aureus strain 502A (ATCC No. 27217; ATCC) or 200 B cepacia bacilli (ATCC No. 25609; ATCC). The number of bacteria in the inoculum was confirmed by serial dilutions and plating. PBS or 5×10⁶ tdTomato-positive GMPs were injected via tail vein immediately after inoculation of bacteria and injection was repeated every 3 days thereafter. Mice were examined daily and euthanized if moribund or 7 days after peritoneal challenge. The presence of intraperitoneal abscesses was assessed by visual inspection. In some experiments, blood cultures were obtained from tail vein blood samples, and bacteremia was quantitated by plate culture.

Medium and reagents. DMEM/F-12 (12400024) and Neurobasal (21103049) media were purchased from Thermo Fisher Scientific. Human insulin (91077C-250MG), human Holo-transferrin (T0665-100MG), putrescine (P5780-5G), sodium selenite (S9133-1MG), linoleic acid (L1012-100MG), DL-alpha tocopherol (vit E, T3251-5G), and bovine serum albumin (A8806-5G) were purchased from Sigma. Recombinant murine SCF (250-03), recombinant human M-CSF (300-25), and recombinant human G-CSF (300-23) were purchased form PeproTech. GDC-0879 (S1104) and SKL2001 (S8302) were purchased from Selleck.

To prepare B7 medium, 500 ml of DMEM/F-12 and 500 mL of Neurobasal media were mixed and supplemented with 4 mg human insulin, 20 mg human Holo-Transferin, 16 mg putrescine, 12.5 μg sodium selenite, 1 mg linoleic acid, 1 mg vit E, and 2.5 g bovine serum albumin. Insulin does not dissolve readily; dissolve insulin in sterile 0.01 M HCl overnight at 4° C. to produce a 10 mg/mL stock solution. Store in 1-mL aliquots at −20° C. The suspension was mixed well before aliquoting.

Mouse GMP derivation, expansion, and differentiation. Cells were cultured at 37° C. in a 5% CO₂ water jacket incubator (Thermo Scientific). Bone marrow cells isolated from C57BL/6J, mTmG or CAG-Cas9-EGFP mice were plated into 6-well plates at a density of 2×10⁶ cells/well and cultured in 2 mL B7 medium supplemented with 50 ng/mL SCF, 1 μM GDC-0879, and 10 μM SKL2001 (SCF/2i). After 3-4 days, cells were dissociated into single-cell suspension by pipetting up and down and replated into 6-well plates at a density of 2×10⁶ cells/well and cultured in 2 mL B7 medium supplemented with SCF/2i. After 2 passages in SCF/2i, the majority of cells were GMPs. GMPs were routinely passaged every 3 days. To induce differentiation, GMPs were plated into 10 cm tissue culture dishes and cultured in RPMI-1640 medium containing 10% FBS and supplemented with either 20 ng/mL M-CSF (for macrophage differentiation) or 20 ng/mL G-CSF (for granulocyte differentiation). GMP-derived macrophages were harvested on day 7 (medium was changed once on day 4) and GMP-derived granulocytes were harvested on day 3 and used for the further experiments.

To generate bone marrow-derived macrophages, 2×10⁶ bone marrow cells isolated from the C57BL/6J mouse were plated into a 10 cm tissue culture dish and cultured in RPMI-1640 medium containing 10% FBS and 20 ng/ml M-CSF. The medium was changed on day 4 and cells were harvested on day 7.

Peritoneal macrophages were generated by injection of 1 mL of 2% Bio-Gel P-100 (Bio-Rad, 1504174) into the mouse peritoneal cavity immediately after transplantation of tdTomato-positive GMPs, followed by peritoneal lavage with sterile PBS 4 days later. Cells collected from the peritoneal cavity were used for fluorescence imaging and flow cytometry analysis.

GMP cell derivation and expansion. Cord blood samples were obtained from StemCyte (Baldwin Park, CA), whole bone marrow was purchased from Stemcell Technologies (Cat #70502.2) and mobilized peripheral blood was purchased from StemExpress (Cat #MLE4GCSF5). Mononuclear cells were isolated using the Ficoll-Paque™ PLUS kit (GE Healthcare Life Sciences, 17-1440-03). Briefly, the blood was diluted with PBS at 1:3 ratio and added into SepMate™-50 tubes (Stemcell Technologies, 85460) preloaded with 15 ml Ficoll-Paque™ PLUS. After centrifugation at 1200 g for 20 minutes at room temperature, the top layer was collected and centrifugated at 300× g for 10 minutes at 4° C. The residual red blood cells were removed by using ACK lysing buffer. Cells were used immediately or cryopreserved in liquid nitrogen.

For the expansion of GMPs, Lin⁻ (CD3, CD14, CD19 and CD56) CD34⁺CD38⁺CD45RA⁺ GMPs were sorted from mononuclear cells isolated from cord blood, whole bone marrow or mobilized peripheral blood. Sorted GMPs were plated into 96-well plates at a density of 4×10⁴ cells/well and cultured in B6 medium supplemented with SCF (50 ng/mL. AF-300-07, PeproTech), GDC-0879 (1 μM).

Five days after the initial plating, GMPs were routinely passaged every 3 days by re-plating them into 48-well plates at a density of 1×10⁵ cells/well and cultured in the modified SCF/2i. Replacement of GDC-0879 with SB590885 (0.5 μM. S2220, Selleck) could slightly increase GMP proliferation rate. To prepare B6 medium, 500 mL of DMEM/F-12 and 500 ml of Neurobasal media are mixed and supplemented with 4 mg insulin, 20 mg Holo-Transferrin, 12.5 μg sodium selenite, 1 mg linoleic acid, 1 mg vit E, and 2.5 g serum albumin.

Human leukemia cell derivation. Clinical specimens were obtained from adult B-cell acute lymphoblastic leukemia (B-ALL) patients. Human B-ALL cells were isolated from B-ALL patients' bone marrow aspirates by sorting for human CD45⁺ and CD19⁺ cells. Human B-ALL cells were transduced with GFP lentivirus. Cells were transplanted into NSG mice, and GFP⁺ leukemia cells were sorted from mouse spleens 6 weeks after transplantation.

Chimeric antigen receptor for macrophage phagocytosis (CarP). The CarP constructs used in mouse GMPs were constructed by fusing human CD19 scFv or HER2 scFV to the human CD8 hinge and transmembrane region and linked to P2A-RFP (CarP-RFP), mouse Fcer1g (NM_010185.4, aa19-86)-mouse CD19 (NM_009844.2, aa491-535)-P2A-RFP (CarP^{Fc19}-RFP), or mouse CD3ζ (NM_001113391.2, aa52-164)-mouse Fcer1g (aa45-86)-mouse CD19-P2A-RFP (CarP^{zFc19}-RFP). The intracellular domain of the αCD19 CarP construct used in GMPs was human CD3ζ (NM_198053.2, aa52-164)-human Fcer1g (NM_004106.1, aa45-86)-human CD19(NM_001178098.1, aa498-544). All CarP receptors contain an N-terminal CD8a signal peptide (MALPVTALLLPLALLLHAARP (SEQ ID NO:1)) for membrane targeting. All the receptors were codon optimized, synthesized by Integrated DNA Technologies and cloned into a modified pSin-EF2 lentiviral backbone by restricting enzyme cutting and T4 ligation.

Electroporation, lentivirus production and GMP transduction. GFP mRNA (TriLink, L7601-100) and single guide-RNA (Synthego, sequence: CCGUCCAGCUCGACCAGGAU (SEQ ID NO:2)) were electroporated into GMPs using the Neon transfection system (ThermoFisher, MPK5000). Briefly, GMPs derived from WT or CAG-Cas9-EGFP mice were expanded in SCF/2i. For transfection, GMPs were harvested and washed twice with PBS and resuspended in buffer R at a concentration of 1×10⁷/mL. 5 μg GFP mRNA or sgRNA was add into 10 μl suspension of WT or CAG-Cas9-EGFP GMPs, and electroporated at 1600V 20 ms 1 pulse. After electroporation, GMPs were plated and cultured in SCF/2i. 48 hours later, GFP expression was examined using fluorescence microscopy and flow cytometry.

Lentivirus was produced by co-transfection of pSin plasmids and vectors encoding packaging proteins (pSPAX and pVSVG) using lipofectamine LTX with plus transfection reagent (ThermoFisher, 15338100) in Lenti-X 293T cells (Takara, 632180) plated in 10 cm dishes at approximately 80% confluence. Viral supernatants were collected 2 days after transfection, 0.45 μM filtered and concentrated with Lenti-X concentrator (Takara, 631232). Concentrated viruses were used for transduction immediately or frozen for long term storage. For GMP transduction, lentivirus was added to GMP cultures and centrifuged at 800 g for 1.5 hours at 32° C. Cells were resuspended in fresh medium and cultured for 48 hours. RFP-positive cells were sorted by FACS.

Quantification and Statistical Analysis. Statistical analysis (excluding RNA-seq experiments) was conducted using the PRISM program (GraphPad). Two groups were compared using an unpaired t test. To assess the statistical significance of differences between more than two treatments, two-way ANOVA was utilized.

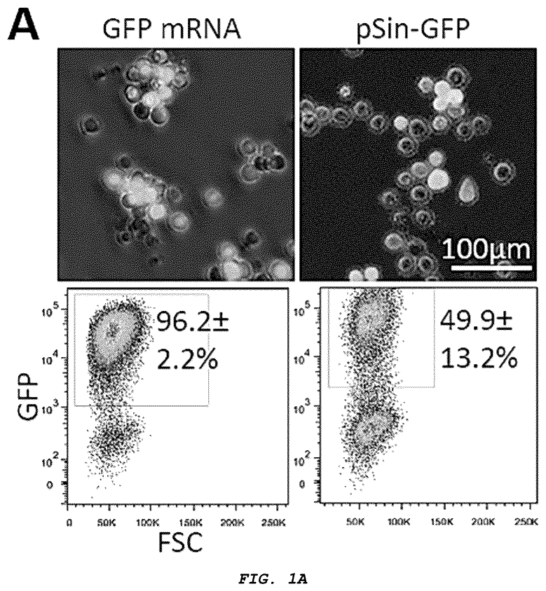

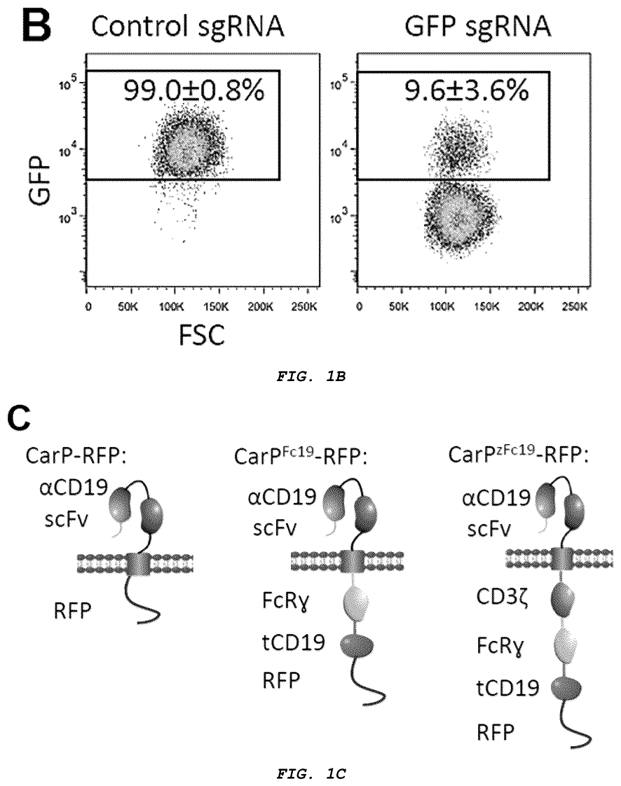

Genetic engineering of SCF/2i GMPs to selectively target cancer cells. Macrophages are an attractive therapeutic target to treat cancer. Macrophages exhibit their anticancer effect through phagocytosis of cancer cells and subsequent presentation of cancer antigens to T cells. Since macrophages are hard-to-transfect cells, it was assessed whether genetic engineering can be performed on SCF/2i GMPs, and whether macrophages from these genetically engineered GMPs could be used to selectively target cancer cells. First, it was demonstrated that high efficiencies of gene modification could be achieved in SCF/2i GMPs. Next, as a proof-of-principle study, GMPs were genetically engineered to specifically target human B cell lymphoma. Chimeric antigen receptor (CAR) T cell therapies have been approved by the U.S Food and Drug Administration (FDA) to treat B cell lymphoma. More recently, studies have demonstrated that macrophage-mediated phagocytosis of cancer cells can be enhanced through engineering macrophages to express a CAR for phagocytosis (CarP). A CarP was generated containing the extracellular single-chain antibody variable fragment (scFv) that recognizes human B cell antigen CD19 (αCD19 scFv), the human CD8 transmembrane domain, and the mouse CD19 cytoplasmic domain fused with the mouse common γ subunit of Fc receptors (FcRγ). This CarP^{Fc19} transgene was linked to the Red Fluorescent Protein (RFP) (CarP^{Fc19}-RFP) to facilitate monitoring of transgene expression. A control CarP was constructed containing the extracellular αCD19 scFv antibody fragment, the CD8 transmembrane domain, and a cytoplasmic RFP, but without the cytoplasmic signaling domain (CarP-RFP) (see FIG. 1C).

After introducing CarP^{Fc19}-RFP and CarP-RFP into SCF/2i GMPs by lentiviral infection, RFP positive GMPs were sorted and expanded in SCF/2i. To evaluate phagocytosis, macrophages were generated from CarP^{Fc19}-RFP and CarP-RFP GMPs and co-cultured with GFP-labeled human B-cell acute lymphoblastic leukemia (B-ALL) cells. As expected, very rare (0.21±0.08%) phagocytosis was observed in the CarP-RFP group, as CarP-RFP lacks the cytoplasmic domain responsible for activating phagocytosis signal. In contrast, 5.23±1.32% of CarP^{Fc19}-RFP macrophages engulfed GFP-positive human B-ALL cells within 1 hour of co-culture (see Figure S2A).

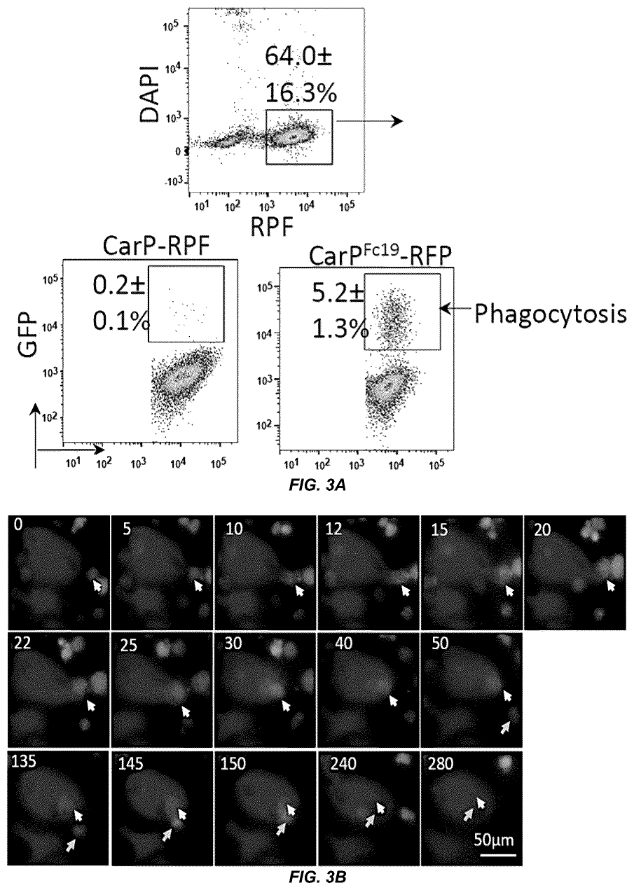

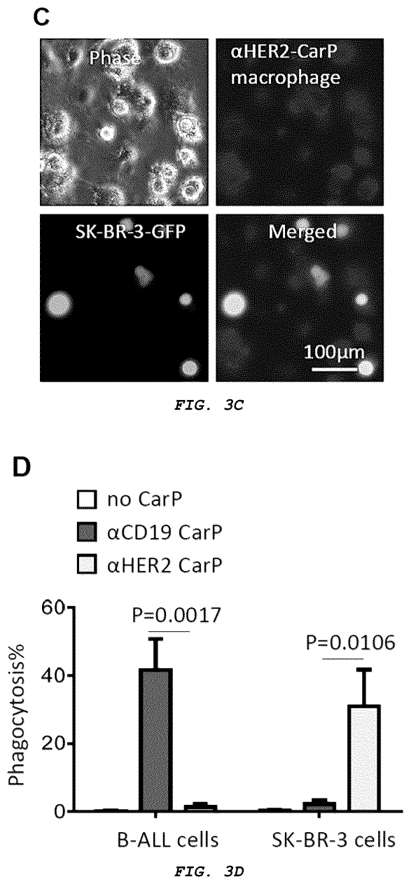

Because the phagocytosis efficiency of macrophages expressing CarP^{Fc19}-RFP was still relatively low, it was tested whether the efficiency could be improved by combining signaling motifs that can promote phagocytosis. Since CD3ζ intracellular domain contains the same immunoreceptor tyrosine-based activation motif (ITAM) as FcRγ, and has been shown to be able to enhance phagocytosis (Isakov, 1997), the cytoplasmic domain of CarP^{Fc19}-RFP was modified by adding the mouse CD3ζ cytoplasmic domain (CarP^{zFc19}-RFP) (see FIG. 1C). When co-cultured with human B-ALL cells, CarP^{zFc19}-RFP-expressing macrophages immediately started to engulf leukemia cells. Some macrophages phagocytized multiple leukemia cells (see FIG. 1D and FIG. 3B). Flow cytometry analysis showed that 41.57±9.26% of CarP^{zFc19}-RFP-expressing macrophages engulfed leukemia cells within 1 hour of co-culture (see FIG. 1E).

Next, the specificity of CarP macrophages in targeting cancer cells was evaluated. The αCD19 scFv cassette of the CarP^{zFc19}-RFP was replaced with the human epidermal growth factor receptor 2 (HER2) scFv to generate the αHER2 CarP. αHER2 CarP macrophages generated from αHER2 CarP GMPs were co-cultured with GFP-labeled SK-BR-3 cells, a human breast cancer cell line that overexpresses HER2. 30.8±6.3% αHER2 CarP macrophages engulfed SK-BR-3-GFP cells within 1 hour of co-culture, whereas phagocytosis was very rare when αHER2 CarP macrophages were co-cultured with GFP-labeled human B-ALL cells that do not express HER2 (see FIG. 3C-D). In contrast, CarP^{zFc19}-RFP macrophages efficiently engulfed CD19-expressing human B-ALL cells, but not SK-BR-3 cells which are CD19 negative (see FIG. 3D). These results suggest that CarP macrophages target cancer cells in a highly specific manner.

CD47 blockade synergistically enhances phagocytosis of CarP macrophages. Previous work suggested that macrophage phagocytic efficiency could be increased by blocking CD47, the macrophage “do not eat me” signal. It was tested whether CarP^{zFc19} and anti-CD47 antibody could act synergistically to enhance macrophage phagocytosis. Within 1 hour of co-culture, 86.2±13.8% of CarP^{zFc19}-RFP-expressing macrophages engulfed human B-ALL cells pre-incubated with 20 μg/ml anti-CD47 antibody for 30 minutes, as compared to 41.6±9.3% without pre-incubation. For macrophages expressing CarP-RFP, phagocytosis efficiency increased from 0.21±0.08% to 18.57±2.85% when human B-ALL cells were pre-incubated with anti-CD47 antibody. More significantly, nearly all human B-ALL cells pre-incubated with anti-CD47 antibody were engulfed and digested by CarP^{zFc19}-RFP-expressing macrophages after 24 hours of co-culture. These data demonstrate that CarP and anti-CD47 antibody act synergistically to improve macrophage phagocytosis of cancer cells.

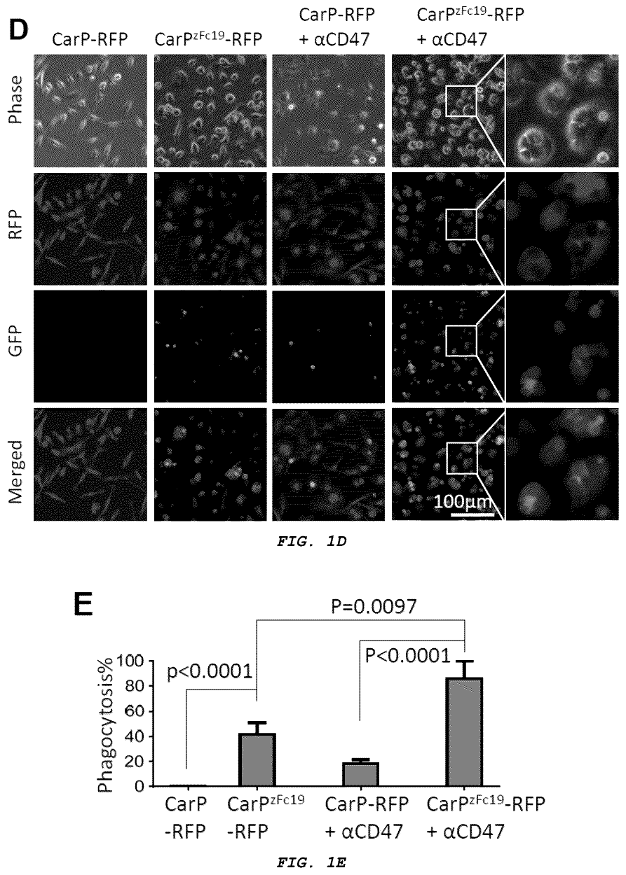

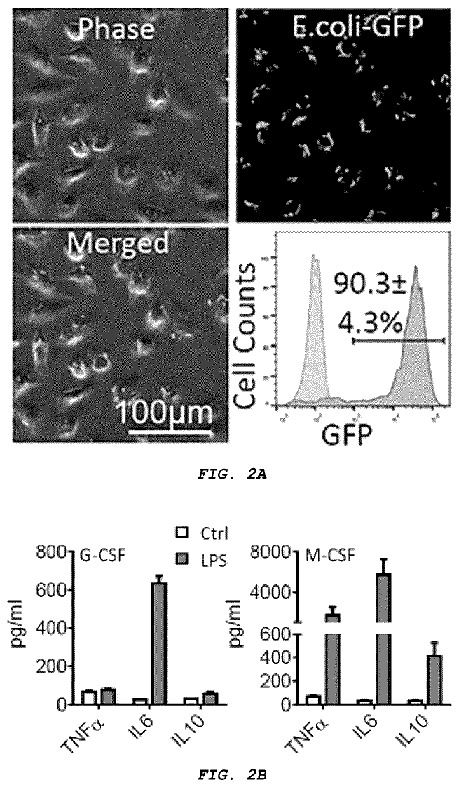

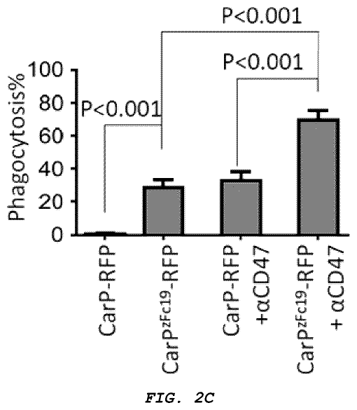

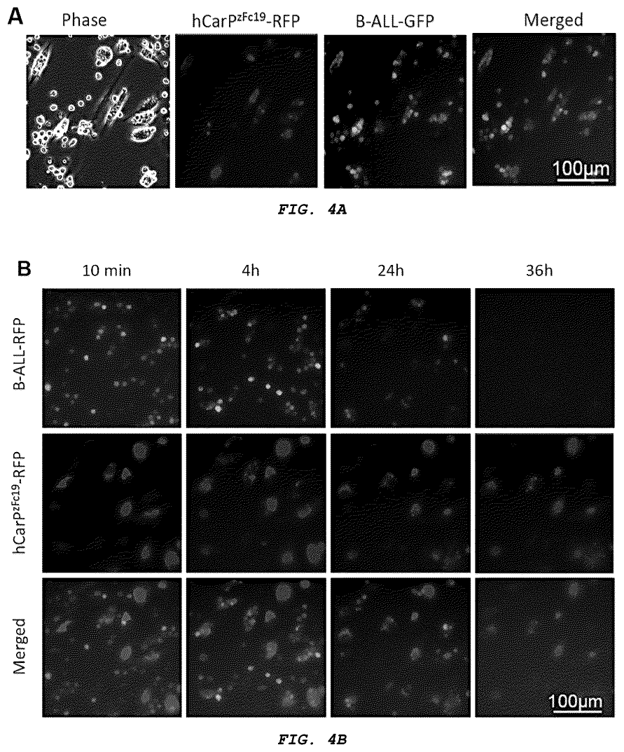

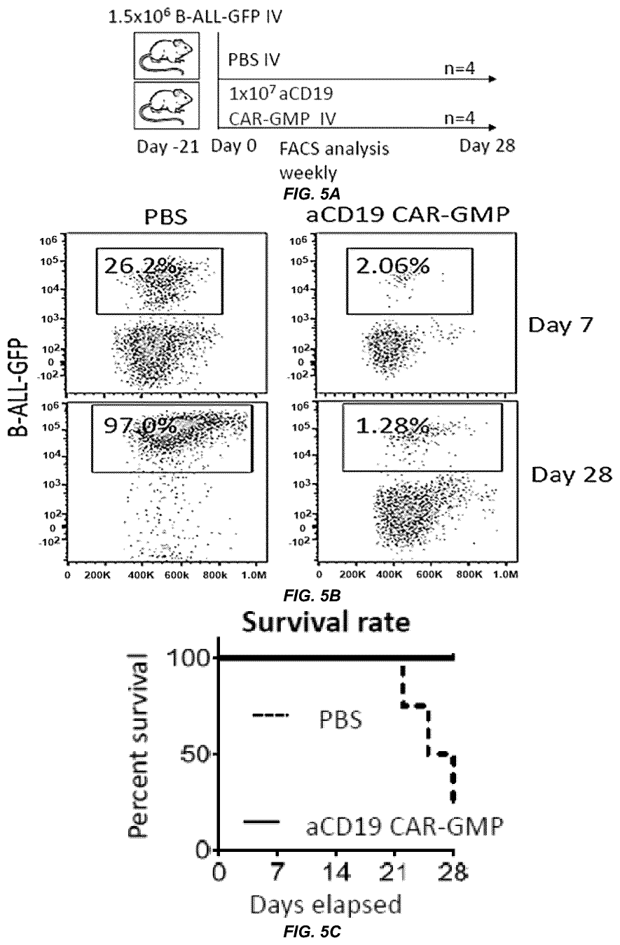

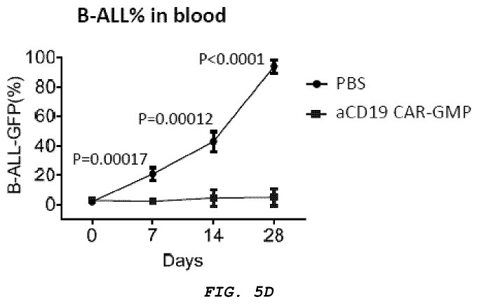

Engineering ex vivo expanded GMPs to selectively target cancer cells. To determine whether ex vivo expanded GMPs could be genetically engineered to selectively phagocytize human B-ALL cells, CarP^{zFc19}-RFP-expressing GMPs were generated and expanded in the modified SCF/2i. To evaluate phagocytosis, macrophages generated from CarP^{zFc19}-RFP GMPs were co-cultured with GFP-labeled human B-ALL cells. Flow cytometry analysis showed that 28.6±4.5% of CarP^{zFc19}RFP-expressing macrophages engulfed leukemia cells within 1 hour of co-culture, compared to 0.87±0.2% of macrophages expressing CarP-RFP (see FIG. 2C and FIG. 4A). The phagocytosis efficiencies for CarP^{zFc19}-RFP and CarP-RFP macrophages were further increased to 69.5±5.6% and 32.8±5.5%, respectively, when human B-ALL cells were pre-incubated with anti-CD47 antibody (see FIG. 2C). More significantly, CarP^{zFc19}-RFP macrophages engulfed and digested nearly all human B-ALL cells pre-incubated with anti-CD47 antibody after 36 hours of co-culture (see FIG. 4B).

It will be understood that various modifications may be made without departing from the spirit and scope of this disclosure. Accordingly, other embodiments are within the scope of the following claims.

1. A method to genetically engineer granulocyte-macrophage progenitors (GMPs) to express a chimeric antigen receptor (CAR) comprising:
introducing a vector comprising a CAR into GMPs to form GMPs that express CAR (CAR-GMPs);
expanding and culturing the CAR-GMPs for multiple passages in defined culture conditions to generate a population of CAR-GMPs; and
inducing the population of CAR-GMPs to differentiate into granulocytes, macrophages or dendritic cells in vitro, wherein the granulocytes, macrophages or dendritic cells express CAR.

2. The method of claim 1, wherein the GMPs are obtained from stem cells.

3. The method of claim 2, wherein the stem cells are hematopoietic stem cells.

4-6. (canceled)

7. The method of claim 1, wherein the CAR comprises an extracellular domain capable of binding to an antigen, a transmembrane domain and at least one intracellular domain that is designed to increase the anti-tumor activities of granulocytes, macrophages and dendritic cells by increasing their phagocytosis and/or proinflammatory cytokines secretion.

8. The method of claim 1, wherein the vector is a viral vector.

9. The method of claim 8, wherein the viral vector can be replicating or non-replicating, and can be an adenoviral vector, an adeno-associated virus (AAV) vector, a measles vector, a herpes vector, a retroviral vector, a lentiviral vector, a rhabdoviral vector, a reovirus vector, a Seneca Valley Virus vector, a poxvirus vector, a parvovirus vector, or an alphavirus vector.

10. (canceled)

11. The method of claim 1, wherein the defined culture conditions include culturing the CAR-GMPs in a culture medium comprising:
(i) a growth factor,
(ii) a B-Raf kinase inhibitor, and
(iii) a Wnt activator and/or a GSK-3 inhibitor,
wherein the CAR-GMPs remain substantially morphologically unchanged after undergoing multiple cell passages and/or clonal expansion.

12. The method of claim 11, wherein the culture medium comprises DMEM/F12 and Neural Basal Medium.

13. The method of claim 12, wherein the culture medium comprises DMEM/F12 and Neural Basal Medium in a ratio of about 5:1 to about 1:5.

14. (canceled)

15. The method of claim 11, wherein the culture medium comprises one or more supplements selected from insulin, transferrin, bovine serum albumin (BSA) fraction V, putrescine, sodium selenite, DL-α tocopherol, and linolenic acid and/or linoleic acid.

16. The method of claim 15, wherein the culture medium is supplemented with insulin, transferrin, BSA fraction V, putrescine, sodium selenite, DL-α tocopherol, and linolenic acid and/or linoleic acid.

17. The method of claim 11, wherein the growth factor is stem cell factor (SCF).

18. The method of claim 11, wherein the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof.

19. The method of claim 11, wherein the Wnt activator is selected from the group consisting of SKL 2001, BML-284, WAY 262611, CAS 853220-52-7, QS11 and any combination thereof.

20. The method of claim 11, wherein the GSK-3 inhibitor is selected from the group consisting of CHIR99021, CHIR98014, SB216763, BIO, A1070722, AR-A014418 and any combination thereof.

21. The method of claim 1, wherein the CAR-GMPs are induced to differentiate into macrophages comprising:
culturing the CAR-GMPs with a macrophage differentiation medium comprising macrophage colony-stimulating factor (MCSF), wherein the macrophages express a CAR.

22. (canceled)

23. An isolated Macrophage population that express a CAR prepared by a method of claim 21.

24. The method of claim 1, further comprising differentiating the CAR-GMPs into granulocytes comprising: culturing the GMPs with a granulocyte differentiation medium comprising granulocyte colony-stimulating factor (GCSF), wherein the granulocytes express a CAR.

25. (canceled)

26. an isolated Granulocyte population that express a CAR prepared by a method of claim 24.

27. An immunotherapy method for treating a subject having cancer with macrophages or granulocytes that express a chimeric antigen receptor (CAR):
administering a composition comprising the macrophages of claim 21 or the granulocytes of claim 24 to the subject having cancer.

28-30. (canceled)
